(19) United States
(12) Patent Application Publication
(10) Pub. No.: US 2024/0293082 A1
(43) Pub. Date: Sep. 5, 2024

(54) INTEGRATED DATA COLLECTION DEVICES FOR USE IN VARIOUS THERAPEUTIC AND WELLNESS APPLICATIONS

(71) Applicant: Cybin IRL Limited, Dubllin (IE)

(72) Inventors: Brett J. Greene, Fitchburg, MA (US); Adu Matory, Berlin (DE)

(73) Assignee: Cybin IRL Limited, Dublin (IE)

(21) Appl. No.: 18/576,487

(22) PCT Filed: Jul. 8, 2022
(86) PCT No.: PCT/EP2022/069109

§ 371 Date: Jan. 4, 2024

Related U.S. Application Data
(60) Provisional application No. 63/219,880, filed on Jul. 9, 2021.

(51) Int. Cl. A61B 5/00; A61B 5/0205; A61B 5/024; A61B 5/16; A61B 5/398; G16H 20/70; G16H 40/67

(52) U.S. Cl. CPC A61B 5/486; A61B 5/0205; A61B 5/165; A61B 5/6803; A61B 5/7267; A61B 5/742; A61B 5/7475; G16H 20/70; G16H 40/67; A61B 5/0075; A61B 5/02416; A61B 5/163; A61B 5/398; A61B 5/4803; A61B 5/4839; A61B 5/4848

(57) ABSTRACT

Systems and methods are described that provide integrated user data collection devices for use in various therapies. The systems comprise a grip device with buttons to detect pressing patterns of a user. a mask device with one or more functional near infrared spectroscopy sensors, and a wearable device with biometric sensors. Alternatively. a camera module may be provided to record a video feed. The system is configured to transmit the data to a user device, which in turn is configured to provide a playback interface displaying the collected data. A device with a microphone, a speaker and an LED configuration illuminating a pattern based on a neurofeedback signal is also provided.

### CROSS-REFERENCE TO RELATED APPLICATIONS

This application claims priority to U.S. Provisional Application No. 63/219,880, filed Jul. 9, 2021, which is herein incorporated by reference in its entirety.

### BACKGROUND OF THE DISCLOSURE

Psychedelic compounds, both natural and synthetic, such as tryptamines, phenethylamines, ergolines, and other derivatives, possess a range of valuable therapeutic properties that can be useful in the treatment of a variety of central nervous system and mental health disorders (e.g., depression, PTSD, OCD, addiction, etc.) and other diseases, especially when administered with therapeutic intention in a therapeutic context (known colloquially as “set and setting”). Psychedelic-assisted psychotherapy involves administering psychedelic drugs to a patient, while a therapist monitors and supports the patient during the session (i.e., the “trip”) as necessary. Psychedelic-assisted psychotherapy in a clinical context typically includes pre-and post-trip therapy sessions to prepare for and evaluate/reflect on the psychedelic experience. Positive patient outcomes of psychedelic-assisted psychotherapy can arise from the altered cognitive and emotional states that accompany the trip, in combination with set and setting. Positive outcomes reported from psychedelic-assisted psychotherapy include the curing of severe post-traumatic stress disorder with MDMA therapy and the cessation of nicotine use by long term tobacco users with psilocybin-assisted psychotherapy. Psychedelic Integration, or the process of psychologically integrating the insights and resolving the challenges of a psychedelic experience, is widely regarded as essential. Until recently, however, efforts to design and validate integration practices in peer reviewed scientific journals have been scant.

### SUMMARY OF THE DISCLOSURE

A system for data collection during therapy on a patient can include a grip device to be held by the patient, the grip device comprising one or more buttons and being configured to detect pressing of a pattern of the one or more buttons by the patient; and transmit an indication of the pattern to a user device. The system can further include a mask device to be positioned over eyes of the patient, the mask device comprising one or more functional near infrared spectroscopy (fNIRS) sensors and being configured to measure fNIRS data from the patient; and transmit the fNIRS data to the user device. The system can further include a wearable device to be worn by the patient, the wearable device being configured to measure biometric data from the patient; and transmit the biometric data to the user device. The user device can be configured to provide a playback interface displaying at least one of the fNIRS data or biometric data.

In some embodiments, the system can include a provider device communicably coupled to the user device and configured to provide the playback interface. In some embodiments, the biometric data can include at least one of a heart rate, an average body temperature, motion information, oxygen levels, respiratory rates, blood pressure, or skin moisture information. In some embodiments, the mask device can include at least one microphone and can be configured to measure audio data from the patient; and transmit the audio data to the user device to be displayed in the playback interface. In some embodiments, the user device can be configured to generate a transcription based on the audio data and display the transcription in the playback interface.

In some embodiments, the user device can be configured to perform vocal analysis and sentiment analysis on the audio data and quantify a mood assessment for the patient. In some embodiments, the system can include a server communicably coupled to the user device. The server can be configured to receive the audio data; generate a transcription based on the audio data; and transmit the transcription to the user device. In some embodiments, the system can include a server communicably coupled to the user device. The server can be configured to receive the audio data; perform vocal analysis on the audio data; perform sentiment analysis on the audio data; quantify a mood assessment for the patient; and transmit the mood assessment to the user device.

In some embodiments, the playback interface can include a linear editor and is configured to receive a selection of at least one data stream; and play the at least one selected data stream in synchronization on the user device. In some embodiments, the system can include a server communicably coupled to the user device. The server can be configured to receive the biometric data and the fNIRS data; and analyze the biometric data and the fNIRS data to detect, via a machine learning algorithm, at least one timepoint. In some embodiments, the user device can be configured to detect, via a machine learning algorithm, at least one timepoint in the at least one data stream. In some embodiments, the playback interface can be configured to receive an annotation for the at least one timepoint.

In some embodiments, the system can include a server communicably coupled to the user device. The server can be configured to receive the biometric data and fNIRS data from the user device; identify a previously recorded data stream associated with the patient; and execute a neurofeedback procedure on the received data and the identified data stream. In some embodiments, the server can be configured to, in response to the execution of the neurofeedback procedure, transmit one or more feedback signals to the user device. In some embodiments, the execution of the neurofeedback procedure can be performed based on the pattern of the one or more buttons. In some embodiments, the user device can include a data hub configured to store baseline data of the patient and the user device is configured to compare the fNIRS data and biometric data to the baseline data.

According to another aspect of the present disclosure, a method for administering a therapy on a patient can include receiving fNIRS data measured by a mask device positioned over eyes of the patient; receiving biometric data measured by a wearable device worn by the patient; receiving audio data measured by at least one microphone positioned at a head of the patient; and providing a user-configurable playback interface displaying at least one of the fNIRS data or biometric data. In some embodiments, the method can include analyzing the biometric data and the fNIRS data, via a machine learning algorithm, to detect at least one timepoint. In some embodiments, the method can include receiving, via the playback interface, an annotation for the at least one timepoint.

In some embodiments, the method can include performing vocal analysis on the audio data; performing sentiment analysis on the audio data; quantifying a mood assessment for the patient; and displaying the mood assessment via the playback interface.

According to another aspect of the present disclosure, a device for collecting data from a patient during a therapy can include a microphone configured to record patient audio; a speaker configured to play audio; one or more functional near infrared spectroscopy (fNIRS) sensors; and a light emitting diode (LED) configuration configured to illuminate a pattern based on a received neurofeedback signal. In some embodiments, the device can be configured to be worn around a head of the patient. In some embodiments, the device can include a zippable functionality, wherein the device is configured to operate in conjunction with a virtual reality headset. In some embodiments, the device can include a nebulizer extension for drug delivery and configured to collect dose data. In some embodiments, the device can include one or more pupillometric sensors configured to measure pupil dilation information from the patient. In some embodiments, the device can include one or more electrooculography (EOG) sensors configured to detect eye movement of the patient. In some embodiments, the device can include a camera module configured to record a video feed of a face of the patient.

According to another aspect of the present disclosure, a system for data collection during therapy on a user can include a grip device to be held by the user. The grip device can include one or more buttons and can be configured to detect pressing of a pattern of the one or more buttons by the user; and transmit an indication of the pattern to a user device. The system can also include a mask device to be positioned over eyes of the user. The mask device can include one or more functional near infrared spectroscopy (fNIRS) sensors and a camera module and can be configured to measure fNIRS data from the user; record a video feed of the user; and transmit the fNIRS data and the video feed to the user device. The user device can be configured to provide a playback interface displaying at least one of the fNIRS data or biometric data.

In some embodiments, the system can include a provider device communicably coupled to the user device and configured to provide the playback interface. In some embodiments, the mask device can include at least one microphone and can be configured to measure audio data from the user; and transmit the audio data to the user device to be displayed in the playback interface. In some embodiments, the user device can be configured to process the video feed to detect pulse data of the user. In some embodiments, the user device can be configured to, in response to detecting pressing of the pattern of the one or more buttons, bookmark a moment in a user data stream.

According to another aspect of the present disclosure, a method for administering a therapy on a user can include receiving fNIRS data measured by a mask device positioned over eyes of the user; receiving a video feed recorded by the mask device; processing the video feed to detect pulse data of the user; receiving audio data measured by at least one microphone positioned at a head of the user; and providing a user-configurable playback interface displaying at least one of the fNIRS data, the audio data, or the pulse data.

In some embodiments, the method can include receiving, via the playback interface, an annotation for at least one section of a data stream, the data stream comprising at least one of the fNIRS data, the audio data, or the pulse data. In some embodiments, the method can include receiving an annotation for at least one section of a data stream, the data stream comprising at least one of the fNIRS data, the audio data, or the pulse data from a provider device. In some embodiments, the method can include receiving an integral from a provider decide on a pre-defined schedule.

### BRIEF DESCRIPTION OF THE FIGURES

FIG. 1 is a block diagram of an example system of integrated data collection for use in therapy or wellness approaches, according to some embodiments of the present disclosure.

FIG. 2 is an example process for enhancing a therapeutic or wellness response via integrated data collection that can be performed within the system of FIG. 1, according to some embodiments of the present disclosure.

FIG. 3 is an example process for generating an integral that can be performed within the system of FIG. 1, according to some embodiments of the present disclosure.

FIG. 4 is an example playback interface, according to some embodiments of the present disclosure.

FIG. 5 is an example neurofeedback technique that can be performed within the system of FIG. 1, according to some embodiments of the present disclosure.

FIG. 6 is another example neurofeedback technique that can be performed within the system of FIG. 1, according to some embodiments of the present disclosure.

FIG. 7 is another example process for generating an integral that can be performed within the system of FIG. 1, according to some embodiments of the present disclosure.

FIG. 8 is an example server device that can be used within the system of FIG. 1 according to an embodiment of the present disclosure.

FIG. 9 is an example computing device that can be used within the system of FIG. 1 according to an embodiment of the present disclosure.

### DESCRIPTION

The following detailed description is merely exemplary in nature and is not intended to limit the invention or the applications of its use.

Therapy is an essential part of psychedelic psychotherapy; it is standard for prescribed psychedelic treatments to include psychotherapy sessions before, during and after a trip. However, it would be desirable to enhance the effectiveness of psychedelic psychotherapy before, during and after a psychedelic experience by using technology to assess patient progress. Data collection during trips and subsequent analytics on the collected data can enhance patient and provider recall of both psychedelic and non-psychedelic therapy sessions. An improved recollection of therapy sessions can lead to improved and sustained recalls of breakthroughs and insights, which can enhance meaningful mental effects and change. Integration of psychedelic experiences is widely acknowledged to be a big part of psychedelic psychotherapy and the ability to recall the experience as greatly as possible may improve one's ability to integrate that experience, extending and maintaining the benefits of an experience, wellness or mindfulness experience/approach. However, current means of preserving session highlights from both psychedelic and non-psychedelic sessions are generally limited to paper and/or electronic note taking by the provider or therapist, or later noted by subjects, with a broad range recall capabilities limiting remembering of details.

In addition, long term psychological assessments of patient data enable more effective and accurate evaluations of psychedelic treatments. Obtaining patient data from treatment sessions can provide valuable context to future psychedelic data interpretations. For example, data from pre-and post-psychedelic therapy sessions enables a more accurate and contextual assessment of psychedelic therapy efficacy that may eventually lead to predictive models for optimizing sessions based on an individual's characteristics. Also, without baseline data, it can be challenging for a therapist to evaluate the short-and long-term success of psychedelic therapy and near-impossible for a drug company to evaluate drug sessions at scale. Therefore, the ability for a data collection system that can integrate with existing and future mental health assessment tools is capable of incorporating long term patient outcome data before and after a psychedelic therapy session, enabling a context for evaluating psychedelic therapy outcomes. Despite the value in patient data from psychedelic experiences, it can also be difficult to collect the actual data from the patients without compromising the therapy itself, which suggests a patient-focused approach to data collection can yield more successful results.

Embodiments of the present disclosure thus relate to a system of integrated data collection devices for use in various therapeutic and wellness techniques, such as psychedelic psychotherapy and especially integration practices following psychedelic and non-psychedelic therapy and wellness sessions. The disclosed system can simultaneously offer improved and more reliable data collection services that collect a wider range of data and an overall improvement to the experience of the patient. In some embodiments, the disclosed system can aid patients and providers in the recall of therapy sessions (both psychedelic and non-psychedelic) by the creation of highlight reels (herein referred to as “integrals”) to aid in the practice of integration. Patients and their providers can use these integrals in coordination with the use of psychedelic treatment and other techniques to improve therapeutic benefits. The disclosed system can utilize various data collection devices, which can each be connected via Bluetooth™ to a user device (e.g., computer, smartphone, etc.) as well as third party applications that can be integrated (e.g. music applications, or behavioral or wellness applications such as Ksana). The data collection devices can include wearable devices to collect biometric data throughout a session, devices facilitating a subject's ability to indicate a moment of interest (bookmark), activate other features, such as a neurofeedback program during a session, or enable non-verbal communication with a provider, such as the need for support, a grip device held in the patient's hand that can monitor grip strength and frequency throughout the session, and a smart mask device to be worn over the patient's eyes during the session. The mask device can simultaneously offer increased comfort and relaxation to the patient (e.g., by playing music via speakers, blocking the patient's vision, offering a facial massage, or exuding heat, coolness, or certain aromas, such as to perform aromatherapy) and valuable data collection services. The blindfold can include a microphone to record vocal and breathing data of the patient, as well as various electrooculography (EOG) detection devices to detect eye movements of the patient, subject, or other user. In addition, the blindfold can include a camera to record video of the patient, as well as various sensors to perform electroencephalogram (EEG) tests ,functional near-infrared spectroscopy (fNIRS) sensors to measure other types of biometric data and LEDs to facilitate a neuro or biofeedback response. In addition, the mask device can include pupillometric detection devices. The data recorded by the devices can be transmitted to the user device or the system server (cloud) and stored for analysis. The disclosed system can also include various modules for performing analysis of the received data, such as vocal analysis, sentiment analysis, mood quantification, and transcription analysis. The disclosed system can compile the analyzed data into a playback engine, which allows both patients and providers (e.g., therapists) to “play back” a psychedelic or non-psychedelic experience that can aid experience recall. Finally, the various components of the disclosed system can be used to implement neuro-and bio-feedback techniques to assist in various therapies and wellness approaches.

It is important to note that the techniques disclosed herein (such as neuro-and bio-feedback) are not limited to psychotherapy but may be used in various types of therapies and have wide-ranging applications. In some embodiments, biofeedback can be used as a diagnostic tool for various neurological disorders, such as through identifying biomarkers consisting of certain patterns of biofeedback performance that may be used for early detection of disease (e.g., Alzheimer's). Biofeedback can also be used for pain reduction. For example, a period where someone with a debilitating condition (e.g., severe depression, anxiety related to cancer, chronic pain, etc.) is experiencing relief from their symptoms can be captured during a session. Then, the person can attempt to synchronize future biometrics to those past biometrics in order to experience symptom relief again. In another example, a palliative technique used by a group of patients suffering from the same issue (e.g., a particular breathing exercise used in a group of people suffering from Lyme disease) can be used in tandem with neurofeedback such that members of the group who have not been able to successfully use the palliative technique to alleviate their issue use biofeedback to synchronize to patients who have successfully used the technique. Feedback techniques can be employed before, during or after a session or desired effects (e.g., such as reducing anxiety or depression).

In some embodiments, biofeedback can be used to develop empathy, such as for people in racial diversity and sensitivity training, those who may be on the autism spectrum, and others experiencing trouble. In some embodiments, biofeedback can be used to develop impulse awareness and control, such as with people who have problems with impulsivity and urge control (e.g., sexual offenders, people with OCD). Here, biofeedback can be used to learn awareness of their biometric states when they are triggered in order to better address these urges as they arise.

In some embodiments, biofeedback can be used to enhance patient care, such as to obtain synchronization between doctors and patients (e.g., in hospice care). In some embodiments, biofeedback can be used to enhance professional training. For example, a student training for a technically demanding profession (e.g., a surgeon) could synchronize with their teacher as they perform the technical skill. In some embodiments, biofeedback can be used to enhance sexual and emotional dynamics, such as in couples' therapy. In some embodiments, biofeedback can be used to enhance skill learning, such as guitar, a language, yoga and meditation techniques, and others. In some embodiments, biofeedback can be used to improve the likelihood of extra-sensory perception between people and to capture talk therapy sessions that do not involve music or psychedelics.

In some embodiments, biofeedback can be used to capture the afterglow of a psychedelic session. For example, the afterglow (a period after the acute effects of a psychedelic pass) can be recorded in a patient undergoing psychedelic-assisted psychotherapy, which can then become the basis for a new type of integral and a go-to “feel good” target biometric state. In some embodiments, musical cues can be used to enhance information retention. For example, a user can record themselves studying while listening to music. The music captured (in the form of integrals) can serve as a memory cue that helps users retain information about what they were studying.

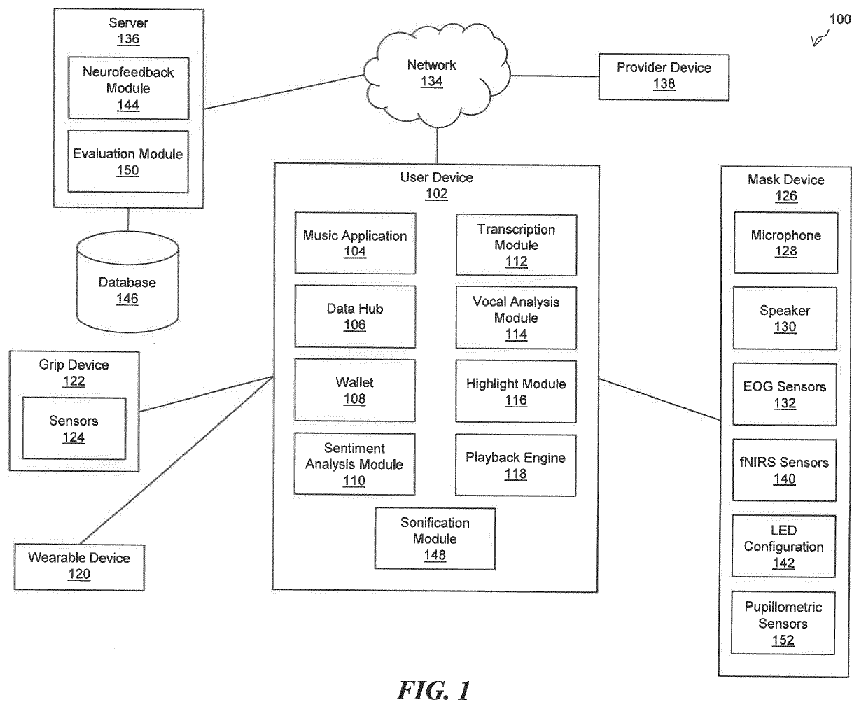

FIG. 1 is a block diagram of an example system 100 of integrated data collection for use in psychotherapy, according to some embodiments of the present disclosure. The system 100 includes a user device 102 (also sometimes referred to as a client device), a provider device 138, and a server 136, which are communicably coupled via a network 134. In addition, user device 102 can be communicably coupled via a Bluetooth™M connection (or other similar near-field connection functionality) to a wearable device 120, a grip device 122, and a mask device 126.

In some embodiments, the system can include any button or series of buttons allowing patients to 1) “bookmark” moments of interest for later playback; 2) non-verbally communicate with providers; 3) activate light within blindfold (including various colors, patterns); and/or 4) activate neurofeedback modes in session (e.g., a calming/mindfulness program for sedation during a challenging moment in a psychedelic assisted or wellness-related experience).

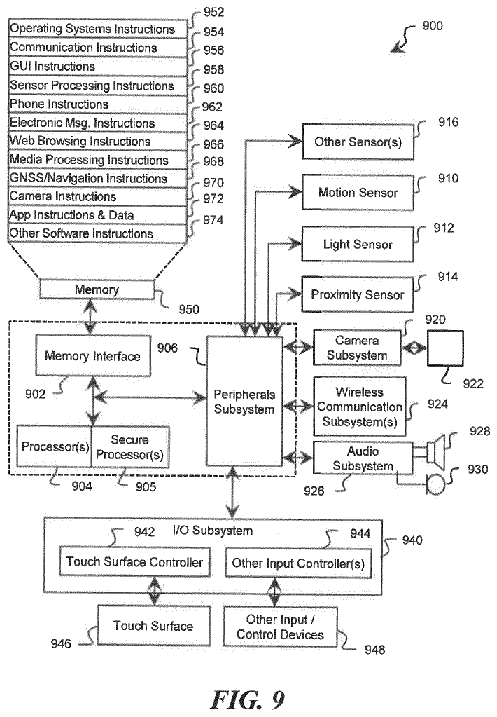

A client device 102 and/or a provider device 138 can include one or more computing devices capable of receiving user input, transmitting and/or receiving data via the network 134, and or communicating with the server 136. In some embodiments, a client device 102 and/or a provider device 138 can be representative of a computer system, such as a desktop or laptop computer. Alternatively, a client device 102 and/or a provider device 138 can be a device having computer functionality, such as a personal digital assistant (PDA), a mobile telephone, a smartphone, or other suitable device. In some embodiments, a client device 102 and/or a provider device can be the same as or similar to the device 900 described below with respect to FIG. 9.

In some embodiments, the system 100 can include any number of client devices 102 and/or provider devices 138 and, for example, the modules 104-118 of the user device can reside in the server 136 and transmission of calculation data and analysis can be transmitted between the server and the user devices 102 and provider devices 138 over the network 134. For example, the server 136 may function as a central processing server for a plurality of user devices 102 associated with a plurality of patients and provider devices 138 associated with a plurality of providers.

In addition, the server 136 can include a neurofeedback module 144. The neurofeedback module 144 is configured to execute neurofeedback/biofeedback techniques and can perform similarity analysis between user biometric data received in real-time (e.g., during a therapy session) and stored user biometric data. In some embodiments, the neurofeedback module 144 can determine discrepancies between the biometric signals and cause the discrepancies to be visualized via numerous visualization modes or filters. In some embodiments, the visualization can occur via the playback engine 118 on the user device 102. Additionally or alternatively, the visualization can occur via the LED configuration 142 of the mask device 126. In the case of LED visualization, the neurofeedback module 144 can determine various parameters that are input to the LED configuration 142, such as color, patterns, timing and frequency of changes, etc. In the case of playback engine 118 visualization, the neurofeedback module 144 can determine various stock images or images with emotional labels (e.g., valence, arousal, etc.) to be displayed. In some embodiments, machine-learning generated images could also be utilized. For example, the visual stimuli can correlate to different stress factors. In some embodiments, the neurofeedback module 144 can also perform audio-based neurofeedback, such as for visually impaired users. Here, the neurofeedback module 144 can determine sounds to be played (e.g., via the user device 102 and/or the speaker 130 of the mask device 126) when the user reaches a level of synchronization or certain biometric levels. In other embodiments, the neurofeedback module 144 can be configured to perform similarity analyses between multiple real-time data signals. For example, the neurofeedback module 144 could receive a stream of biometric/neurometric data both from a user device 102 and a provider device 138 (or other user device 102) and compare these data signals to each other. In this manner, a user can synchronize his/her biometric and neurometric data signals with another user or with a provider.

The server 136 can further include an evaluation module 150. In some embodiments, the evaluation module 150 can include one or more machine learning models that are trained to predict user's therapeutic outcomes and/or subjective experiences. For example, the multimodal data (e.g., biometrics and neurometrics) can be preprocessed and used to train the models. The preprocessing can include dimensionality reduction, feature selection, and inputting missing data. In some embodiments, training the machine learning model(s) can include training on a static data set and/or training in a continual feedback loop, where the model self-optimizes based on discrepancies between its predictions and user-reported outcomes/subjective experiences. Predictions can include general predictions on biometric and neurometric data alone and/or precision models whose performance is optimized by each user's self-reported and baseline data. In some embodiments, predictions can be used during a therapeutic session to improve the user's experience and/or to determine when a patient's symptoms may be recurring.

The network 134 can include one or more wide areas networks (WANs), metropolitan area networks (MANs), local area networks (LANs), personal area networks (PANs), or any combination of these networks. The network 134 can include a combination of one or more types of networks, such as Internet, intranet, Ethernet, twisted-pair, coaxial cable, fiber optic, cellular, satellite, IEEE 801.11, terrestrial, and/or other types of wired or wireless networks. The network 134 can also use standard communication technologies and/or protocols.

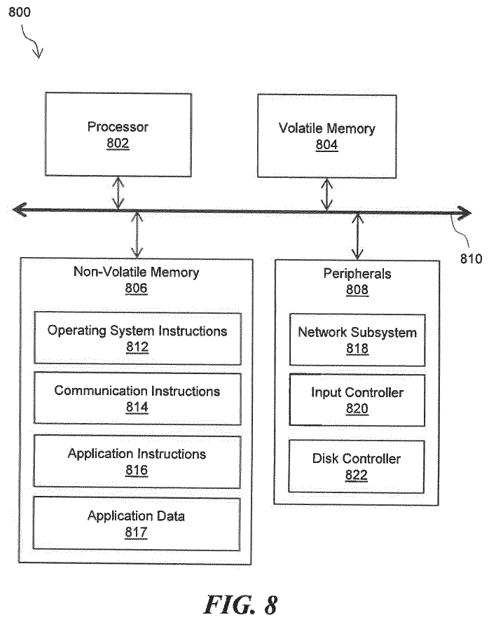

Server device 136 may include any combination of one or more of web servers, mainframe computers, general-purpose computers, personal computers, or other types of computing devices. Server device 136 may represent distributed servers that are remotely located and communicate over a communications network, or over a dedicated network such as a local area network (LAN). Server device 136 may also include one or more back-end servers for carrying out one or more aspects of the present disclosure. In some embodiments, server device 136 may be the same as or similar to server device 800 described below in the context of FIG. 8. In some embodiments, server 136 can include a primary server and multiple nested secondary servers for additional deployments of server 136.

The server 136 can also be communicably coupled to a database 146. In some embodiments, the database 146 can be a GDPR-and HIPAA-compliant database that stores multimodal data in a structured way. The multimodal data can include subjective reports of resonant moments (e.g., bookmarks), biometrics, neurometrics, psychometrics, and baseline data, such as from other apps used by the user).

As shown in FIG. 1, user device 102 can be configured to receive various data from the integrated data collection devices (e.g., wearable device 120, grip device 122, and mask device 126). In some embodiments, wearable device 120 can be a smartwatch, such as an Apple Watch™ or FitBit One™, and can be worn by a user/patient (e.g., around the patient's wrist). The wearable device can include a variety of attachment mechanisms (e.g., clip, clasp, loop, toggle, button, snap, etc.) or adhesive mechanisms. Wearable device 120 can be configured to measure and transmit a variety of biometric/physiological/psychological data to the user device 102 including, but not limited to, heart rate, average body temperature, motion information, oxygen levels, respiratory rates, blood pressure, skin moisture information, etc. In some embodiments, examples of sensors included in the wearable device 120 can include, but is not limited to, one or more of a GPS sensor, accelerometer, directional sensor (e.g., compass), gyroscope, motion sensor, pedometer, passive infrared sensor, ultrasonic sensor, microwave sensor, a tomographic motion detector, a camera, a biometric sensor, a light sensor, a timer, or the like. In some examples, a biometric sensor may include, but is not limited to, one or more health-related optical sensors, capacitive sensors, thermal sensors, electric field (“eField”) sensors, and/or ultrasound sensors, such as photoplethysmogram (“PPG”) sensors, electrocardiogramsors, galvanic skin response (“GSR”) sensors, posture sensors, stress sensors, photoplethysmogram sensor, and the like.

In some embodiments, grip device 122 can be soft and squeezable (e.g., made of a soft material such as silicon) to provide a comforting, passive outlet for the patient to grip and squeeze during a trip or session. In addition, grip device 122 can be configured to, via internal sensors 124, measure and track variations in the grip strength and frequency of a patient throughout the course of a session or trip. In some embodiments, sensors 124 can include various motion and pressure/force-based sensors, including, but not limited to, position sensors, accelerometers, and/or dynamometers. In some embodiments, the grip device 122 can include one or more buttons to facilitate nonverbal communication between a subject and therapist. In response to a button being pushed or certain patterns of buttons being pushed, certain signals can be transmitted to the user device 102 and/or the provider device 138. In some embodiments, the pressing of a button can cause the neurofeedback module 144 to initiate a neurofeedback procedure. In some embodiments, the pressing of a button can trigger a bookmark to be created, demarcating a specific moment in time that the subject deems important.

In some embodiments, mask device 126 can be configured to be worn around a patient's head to cover their eyes and optionally their ears. In some embodiments, mask device 126 can include soft and/or plush materials and padding to increase the comfort of the patient. Mask device 126, when worn by a patient during a trip, can minimize the potential for non-specific psychedelic enhancers that could negatively affect trip outcomes. In some embodiments, mask device 126 can include a microphone 128 that can be configured to record audio from the patient during a trip, such as vocal audio and/or breathing audio. Mask device 126 can be configured to transmit audio recordings to the user device 102. In some embodiments, mask device 126 can also include one or more speakers 130 that can be positioned at or near the patient's ears. The one or more speakers 130 can be configured to play music or music therapy from user device 102, such as music controlled by music application 104. In some embodiments, mask device 126 can include one or more EOG or EEG or FNIR sensors 132. For example, EOG sensors 132 can include a plurality of electrodes (e.g., about 2, 3, 4, 5, 6, 7, 8, 10, 12, 14, 16 or more electrodes) placed at points close to the eyes of the patient (e.g., a first electrode and a second electrode positioned around the eye) and can be configured to measure the electric potential between the electrodes. The variation of the potential can be used to investigate eye movements and blinking activity. The potential between the electrodes approximates the corneo-retinal standing potential that normally exists between the front and back of a human eye. In some embodiments, the electrodes can be placed above and below an eye or to the left and right of the eye. Mask device 126 can then be configured to process (e.g., convert from analog to digital) and transmit the signals to the user device 102 for further analysis (e.g., by a signal processing device, not shown).

In the disclosed embodiments, various data types can be used as a base code for applying various visual rendering algorithms to display the data. This can include fNIRS data, EOG data, EEG data, pupillometry data, and other data streams described herein. For example, the data can be visually rendered in a first-person perspective as part of a simulation of the trip of a patient in a VR immersive environment. Via various algorithms on the user device executed by the processor, a time-series of EOG signals can be analyzed to identify blinks. The identified blinks can be coordinated with blinks of an avatar in the VR environment. In another example, the EOG signals can be correlated with an equalizer band in a screen-saver style program. The equalizer band can be displayed with a variety of filters/effects such as strobe, underwater, outer space, forest, or Alice in Wonderland.

In some embodiments, mask device 126 can further include a camera to record video of the patient. For example, in some embodiments, a video feed of the user may be used to measure certain biometric parameters (e.g., pulse) instead of or in addition to the wearable device 120. Additionally, mask device 126 can include various sensors and circuitry to perform electroencephalogram (EEG) tests. EEG tests can be used to detect electrical activity of a patient's brain using small electrodes attached to the scalp. The mask device 126 can include various electrodes that can attach to the patient's scalp during a trip session, perform EEG tests, record the necessary measurements, and transmit the data to the user device 102 for processing and analysis.

In addition, mask device 126 can have a zippable functionality and can be unzipped to become VR compatible. When unzipped, the EOG sensors 132 can remain intact and the blindfold acts as a compatible extension for a VR headset. The mask device 126 can generally be designed to fit with VR headsets. Mask device 126 can also include ear coverings with speakers for comfortable headphones that fit perfectly with low pressure around the head for a hi fi audio experience.

In some embodiments, nebulizer extension for drug delivery (e.g., psychedelic drug delivery) via inhalation can be integrated with the mask device 126. The nebulizer can collect dose data and provide the dose data to the user device 102 for analysis and playback.

In some embodiments, the mask device 126 can include one or more fNIRS sensors 140 to collect another form of biometric data from the user/subject. The one or more fNIRS sensors 140 can utilize near-infrared spectroscopy to perform functional neuroimaging, such as via measuring oxy- and deoxygenated blood data (i.e., deoxyhemoglobin concentrations), heart rate, and pulse rate variability (herein referred to as “fNIRS data”). In addition, the mask device 126 can include an LED configuration 142, which is configured to display various light signals and patterns in conjunction with the neurofeedback module 144. For example, the LED configuration 142 can be configured to display certain light patterns when a user's biometric data approaches or matches the baseline biometric data its being compared to. This pattern can be determined by either the server 136 or the user device 102. In additional embodiments, the mask device 126 can further include one or more pupillometric sensors 152 configured to measure pupil dilation and other movement.

As shown in FIG. 1, user device 102 can be configured to run various modules and applications. For example, user device 102 can include a music application 104, a data hub 106, a wallet 108, a sentiment analysis module 110, a transcription module 112, a vocal analysis module 114, a highlight or Integral module 116, and a playback engine 118. It is important to note that, in some embodiments, one or more of the modules/applications of user device 102 may, additionally or alternatively, reside on the server 136. Music application 104 can be any standard music-playing application; music has been shown to be effective as a stress and anxiety management tool and has also shown efficacy for diverse outcomes, including chronic and acute pain. Data hub 106 can be configured to capture and aggregate various data streams (e.g., biometric data streams from wearable device 120, grip data from grip device 122, and EOG and audio data from mask device 126) and integrate with various hardware and software plug-ins and IOT devices, such as the other modules in user device 102.

In some embodiments, wallet 108 can be a patient accessible, electronic medical records (EMR) system that provides patients with control on setting permissions for the sharing of their data. The wallet 108 can be HIPAA (Health Insurance Portability and Accountability Act) compliant and can provide secure EMR storage. In some embodiments, wallet 108 can provide functionality for users to monetize their data via providing access to others.

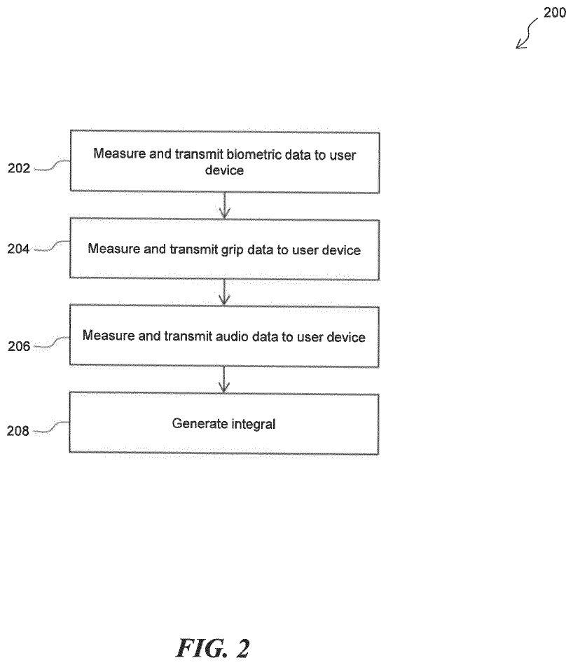

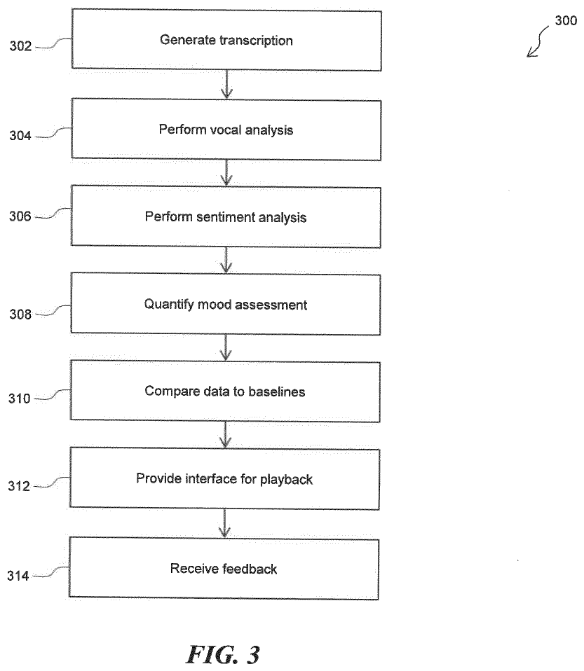

In some embodiments, sentiment analysis module 110 can be configured to perform sentiment analysis on data received from the data collection devices, such as the audio data received from mask device 126. In some embodiments, transcription module 112 can be configured to generate a transcription of the audio recorded by mask device 126 during a session (e.g., via text-to-speech functionality). Further, in some embodiments, the transcription module 112 can be configured to identify specific keywords in a transcription. For example, certain keywords could be defined beforehand by either the subject or the provider. In some embodiments, transcription module 112 can provide transcription results to the sentiment analysis module 110 as an input. In some embodiments, vocal analysis module 114 can be configured to perform acoustic analysis on the recordings to determine various insights based on the tone of the patient. In some embodiments, highlight module 116 can be configured to analyze and compile the various data received from the data collection devices (e.g., the data stored in data hub 106) and generate an integral. As described herein, an integral can refer to artificial intelligence-and machine learning-generated audio-visual compilation of experience highlights rendered from targeted datapoints of the trip session. Additional details on integral generation are described with respect to FIGS. 2 and 3. In some embodiments, playback engine 118 can include a linear editor interface that enables manual playback of audio, visual, transcription, biometric, and other data streams, as well as integrals generated by the highlight module 116. In some embodiments, instances of one or more of the modules and applications of user device 102 can also be on the provider device 138. For example, a playback engine 118 may also run on a provider device so that either the patient/subject/user or the provider can access playback of a trip session when authorized by patient/subject/user. The linear editor allows manual playback; users can select from a multitude of playback interfaces to process and interpret data streams collected within the platform. The linear editor can resemble a digital audio workstation (DAW) or video editor in terms of layout, with each captured data-stream displayed as a separate recorded data stream. Users can also edit their experience, run different filters, etc. The linear editor has both playback and editor/developer functionality. In addition, the playback engine 118 can include a content mode, where programs for replaying data can be accessed and played by users as video, VR, gaming, or app-based mental exercise content.

The various system components—such as modules 104-118—may be implemented using hardware and/or software configured to perform and execute the processes, steps, or other functionality described in conjunction therewith.

FIG. 2 is an example process 200 for enhancing psychotherapy via integrated data collection that can be performed within the system of FIG. 1, according to some embodiments of the present disclosure. For example, process 200 may be performed during psychotherapy (both psychedelic and non-psychedelic) to monitor a patient during a trip. Prior to process 200, the various data collection devices (wearable device 120, grip device 122, and mask device 126) can be positioned according to their respective preferred locations on the patient (e.g., on the patient's wrist, in the patient's hand, and over the patient's ears and eyes, respectively). At block 202, wearable device 120 measures biometric data from the patient over the course of the trip session and transmits the biometric data to the user device 102. In some embodiments, biometric data can include data such as a heart rate, average body temperature, motion information, oxygen levels, respiratory rates, blood pressure, skin moisture information, fNIRS data, etc. At block 204, grip device 122 measures grip data from the patient over the course of the trip session and transmits the grip data to the user device 102. In some embodiments, grip data can include a time-series of grip strength, grip pressure, grip force, etc., and variations thereof.

At block 206, mask device 126, via microphone 128, measures audio data from the patient over the course of the trip session and transmits the audio data to the user device 102. In some embodiments, blocks 202-208 can be performed simultaneously for the duration of a trip session. In some embodiments, music application 104 can cause music to be played during the trip session, cither from the user device 102 itself or via a speaker 130. At block 208, highlight module 116 can generate an integral based on the data received from the various data collection devices. Additional details on the generation of integrals are described in relation to FIG. 3.

FIG. 3 is an example process 300 for generating an integral that can be performed within the system of FIG. 1, according to some embodiments of the present disclosure. In some embodiments, prior to process 300, the measured data that has been transmitted to the user device 102 via the various sensors and integrated devices can be compiled and stored in both data hub 106 and wallet 108. The storage within data hub 106 prepares the data for processing by various additional modules, while the storage in wallet 108 offers a secure, HIPAA-compliant way for a user to manage their own data. At block 302, transcription module 112 generates a transcription of the trip session from the audio recordings. At block 304, vocal analysis module 114 can receive the audio recording data from data hub 106 and perform vocal analysis on the recordings. In some embodiments, vocal analysis module 114 can also analyze the transcription of the audio (e.g., the transcription generated by transcription module 112). At block 306, sentiment analysis module 110 performs sentiment analysis for the user, such as by analyzing the transcription and the audio recording data. This can include natural language processing (NLP) techniques to analyze and interpret the patient's language to determine a “sentiment”. In addition, acoustic analysis can be performed on the recording to make various evaluations based on the tone of the patient (e.g., tonal analysis). This can include analysis of various acoustic properties (e.g., tone, pitch, energy, speaker dominance, silence, cross talk, speech rate, hesitation, pauses, etc.), as well as vocal emotion detection and other emotional indicators. At block 308, highlight module 116 quantifies a mood assessment of the user based on the transcription, the vocal analysis, and the sentiment analysis. In some embodiments, the mood assessment quantification can include NLP techniques to analyze word choices and patterns to derive an emotional and mood assessment. In some embodiments, the mood assessment can be further enriched using acoustic analysis, tonality analysis, and vocal quality.

At block 310, highlight module 116 compares the data measurements to baselines associated with the user. For example, prior to and/or after a trip session where a user consumes psychedelics, the user and his/her therapist may perform one or more baseline sessions, where the various data collection devices (c.g., wearable device 120, grip device 122, mask device 126) measure and record data for the user over a duration of time. This can serve to establish a desirable baseline for various data streams for the user when they are not under the influence of psychedelics. In some embodiments, a “baseline” can refer to recorded heightened experiences to be used at a later point, such as in an attempt to return to the experience via neurofeedback. A baseline can also refer to where a subject is when they are not on a psychedelic and may change over time (e.g., reflecting improved outcomes). Associated integrals can represent personal real world evidence data that users engage with (via neurofeedback and biofeedback) to return to a previously experienced data in a psychedelic/mindfulness/wellness experience. This baseline data can be stored in data hub 106 and wallet 108. By comparing the data obtained from a trip session to baseline data for the user, highlight module 116 (or a provider themselves) can determine variations in user data streams and thus evaluate the effect of the psychedelics on the user. In some embodiments, highlight module 116 can also include various machine learning algorithms to analyze the data streams associated with the user (e.g., any of the data measured including vocal data, biometric data, and grip data). For example, highlight module 116 can detect timepointse.g. the fifteen minute mark, with data streams in aggregate) based on what the provider is looking to target (e.g., difficult moments, transcendent moments, euphoria, relaxation, etc.).

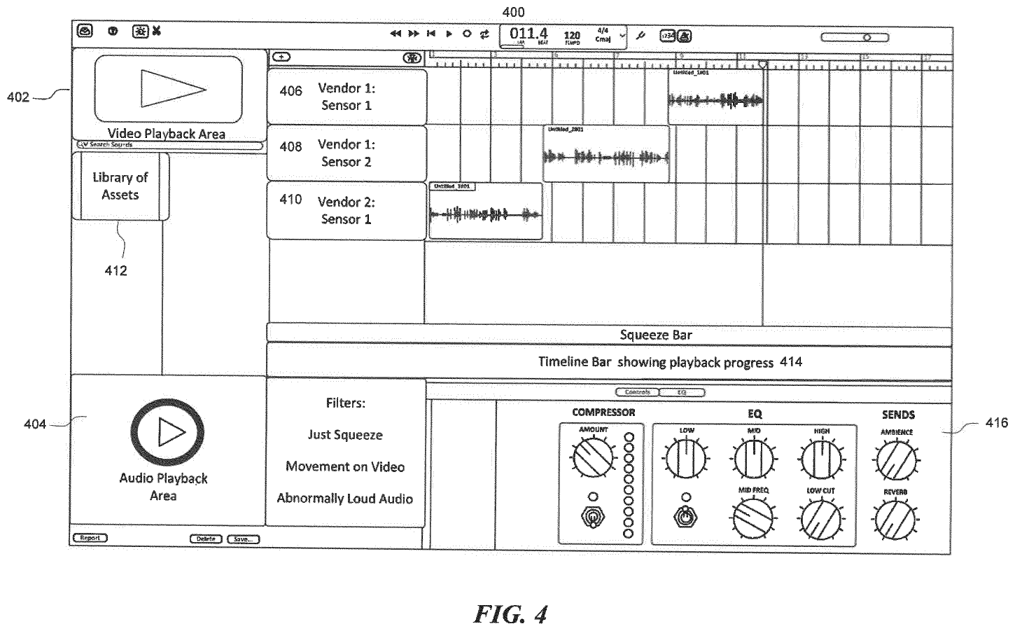

At block 312, playback engine 118 provides an interface for playback. In some embodiments, the playback interface can be displayed on the user device 102, the provider device 138, or both simultaneously; for example, both the patient and the provider may wish to playback the trip session together on their separate devices. In some embodiments, the playback interface can be a linear editor with various tools for viewing and analyzing the data streams from the trip session. The playback interface features montages of audiovisual representations and can be automatically generated with different time intervals, such as 5, 10, or 15 minutes. The playback engine 118 can allow the patient and/or provider to play various data streams from a trip session together in a linear format; additional details of which are discussed below with respect to FIG. 4. At block 314, highlight module 116 can receive feedback. For example, the playback interface can enable patients and/or providers to insert comments or annotations to the streams and highlights. This can enable extra focus to be placed on timepoints detected by highlight module 116. Comments and annotations such as this can further improve the patient's ability to recall the trip session and thus increase the efficacy of the treatment.

FIG. 4 is an example playback interface 400, according to some embodiments of the present disclosure. Playback interface 400 can include a video playback area 402, an audio playback area 404, data streams 406-410, a library of assets 412, a timeline bar showing playback progress 414, and various control tools 416. Playing interface 400 allows a user (which can be either a patient or a provider) to select various data streams via the library of assets 412. By accessing the library of assets 412, the user can access the data hub 106 that maintains the various data obtained from a trip session. This can include grip data, audio data, biometric data, and EOG data. Once the desired data streams are selected, they can be played in a synchronized fashion in the linear editor. For example, a user may wish to fully relive a trip session and may replay the music from the session (e.g., a music data stream), the grip data from the session, the button pushing/bookmarking of a session, or any desired biometric data from the session (heart rate, oxygen levels, temperature, etc.), EOG data from the session, their own audio recordings from the session, and a transcription from the session. These data can be played individually, as individual data streams, altogether all at once, or any combination of the above. This enables a user to visually observe all of the recorded data streams from a trip session behave over time and to identify highlights or determine conclusions. For example, it may be possible to detect that a specific point during a trip session when a word in the music or an uttering by the provider invokes a negative response in the patient by analyzing their data at that time (e.g., spikes in grip force, heart rate, etc.). In some embodiments, various biometric and neuro-metric data streams can undergo a sonification procedure and then be explored during a session replay. Visualization of biometric data streams, such as visualization of inhales and exhales of respiration, enables users to re-synchronize to breathing patterns that they, or another, experienced during a recorded session. Other examples can include pulse, heartrate, EEG signature, FNIRS data, EOG data, or other integrated device measuring biometrics. Neuro and biofeedback can then be employed to allow a subject to synchronize to themself or someone else via one or multiple data streams.

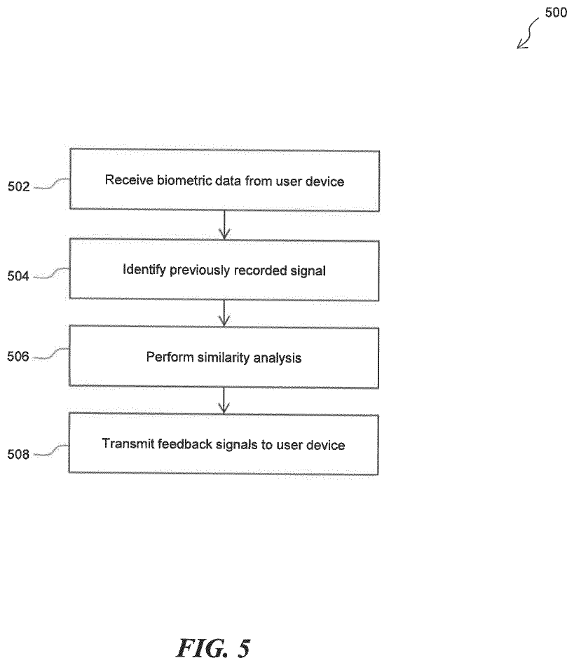

FIG. 5 is an example neurofeedback technique 500 that can be performed within the system of FIG. 1, according to some embodiments of the present disclosure. In some embodiments, technique 500 can be performed by the server 136. At block 502, the server 136 receives biometric data and/or neurometric data from the user device 102. In some embodiments, the received data can include data such as a heart rate, average body temperature, motion information, oxygen levels, respiratory rates, blood pressure, skin moisture information, fNIRS data, etc. At block 504, the server 136 identifies a previously recorded signal associated with the subject. For example, the server 136 can receive a selection of specific time points or sections of a previously recorded session. The selection can be made by a user via a user device 102 or a provider via the provider device 138. In some embodiments, the previously recorded signal can be an integral made by either the subject or the provider. In some embodiments, identifying the previously recorded signal (aka a baseline signal) can include, once the selection has been received, accessing the database 146 to obtain the necessary signals. In some embodiments, the previously recorded signals can include baseline data obtained for the subject via software integrations with various third-party apps, such as Ksana or other baseline data collection apps. This baseline data can include music listening history, geospatial location, screen time on a phone, and information about calls/texts. In addition, the software integration could be with EMR providers.

At block 506, the neurofeedback module 144 performs a similarity analysis on the data received from the user device 102 and the previously recorded signal. In some embodiments, the similarity analysis can include determining discrepancies between the biometric and/or neurometric signals and th previously recorded signal(s). At block 508, the server 136 transmits feedback signals to the user device 102. In some embodiments, the feedback signals can include the discrepancies identified at block 506, which can then be displayed visually on the user device 102, such as via the playback engine 118. In some embodiments, the feedback signals can further include/determine various stock images or images with emotional labels (e.g., valence, arousal, etc.) that have been determined based on the discrepancies, as well as machine-learning generated images. These images can be determined by either the server 136 or the user device 102, in response to receiving the discrepancies. The visual stimuli can correlate to different stress factors. In some embodiments, the neurofeedback module 144 can also perform audio-based neurofeedback, such as for visually impaired users. Here, the neurofeedback module 144 can determine sounds to be played (e.g., via the user device 102 and/or the speaker 130 of the mask device 126) when the user reaches a level of synchronization or certain biometric/neurometric levels. Such audio signals can be included in the feedback signals and transmitted to the user device 102 at block 508.

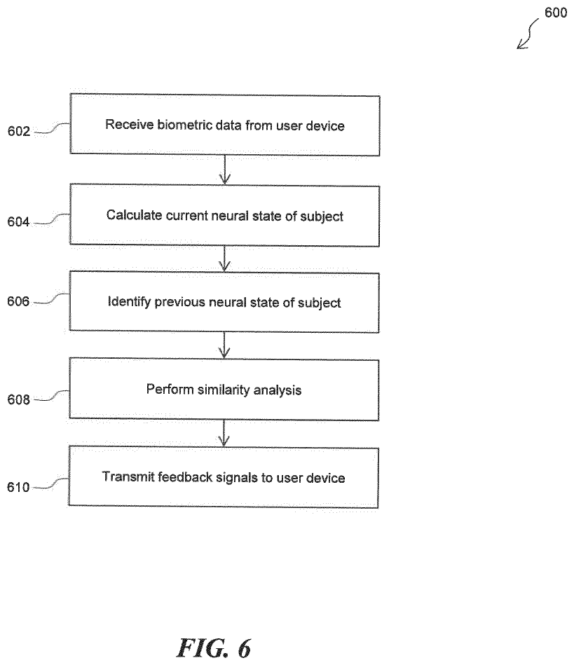

FIG. 6 is another example neurofeedback technique 600 that can be performed within the system of FIG. 1, according to some embodiments of the present disclosure. In some embodiments, technique 600 can be performed by the server 136. At block 602, the server 136 receives biometric data and/or neurometric data from the user device 102. In some embodiments, the received data can include data such as a heart rate, average body temperature, motion information, oxygen levels, respiratory rates, blood pressure, skin moisture information, fNIRS data, etc. At block 604, the neurofeedback module 144 calculates a current neural state of the user based on the received data. Calculating the neural state can include applying one or more Fourier transforms to each of the received data signals and combining the altered signals. Such a neural state can provide more discriminative features allowing for better identification of peak moments of serenity or other emotional peaks.

At block 606, the neurofeedback module 144 identifies a previous neural state of the subject, such as by accessing the database 146. In some embodiments, the previous neural state can be a baseline neural state associated with the subject. In some embodiments, the previous neural state can be associated with either a subjective or objective state of calm. In some embodiments, the neural state can be identified by button push pattern by the subject. For example, the subject may press one or more buttons on the grip device 122 that are indicative of a desired neural state. At block 608, the neurofeedback module 144 performs a similarity analysis on the real-time calculated neural state and the previously stored neural state obtained from the database 146. The similarity analysis can include determining discrepancies between the neural state signals. At block 610, the server 136 transmits feedback signals to the user device 102. The feedback signals can be similar to or the same as those discussed in relation to FIG. 5, where the feedback signals can be displayed directly by the user device 102, can include instructions to be forwarded to the mask device 126 to cause certain illumination patterns by the LED configuration 142, can include instructions for the user device 102 to cause various images to be displayed, and can include instructions for audio-based neurofeedback to be played.

It is important to note that, in some embodiments, a neurofeedback module 144 can also reside on the user device 102 and, therefore, processes 500 and 600 can each alternatively be performed by a user device 102. In such embodiments, the previously recorded signals and other baseline data would be stored and accessed from the data hub 106.

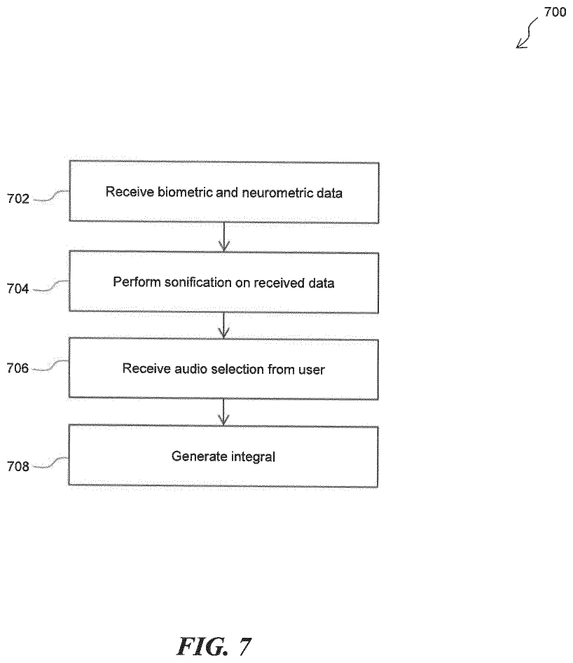

FIG. 7 is another example process 700 for generating an integral that can be performed within the system of FIG. 1, according to some embodiments of the present disclosure. Process 700 can be performed the user device 102. At block 702, the user device 102 receives biometric and neurometric data measured in real-time for a subject. The biometric and neurometric data can have been measured by (and then received from) the mask device 126. In some embodiments, the received data can include data such as a heart rate, average body temperature, motion information, oxygen levels, respiratory rates, blood pressure, skin moisture information, fNIRS data, etc. At block 704, the sonification module 148 performs sonification procedures on the received data. In some embodiments, the sonification procedure can include converting the received data to audible sonic filters. For example, the sonification module 148 can convert the breathing data and fNIRS data to sonic filters, translating the data into pitch, volume, stereo position, etc. At block 706, the user device 102 receives an audio selection from a user. For example, the user can select certain audio clips via the music application 104, such as nature sounds, white noise, etc. In some embodiments, the user can select specific songs via a technical integration with a music API, such as Lucid® or Spotify®. At block 708, the highlight module 116 generates an integral based on the converted biometric and neurometric data signals and the audio selected by the user. The generated integral can then be stored in a database for sharing and can be accessible by various other users via the music application 104. In some embodiments, various algorithms can be used to identify relationships between metadata from music (or other audio clip) and data received on behalf of a subject (biometric or neurometric data). The results could be used to induce a particular emotional state and/or optimize patient outcomes.

FIG. 8 is a diagram of an example server device 800 that can be used within system 100 of FIG. 1. Server device 800 can implement various features and processes as described herein. Server device 800 can be implemented on any electronic device that runs software applications derived from complied instructions, including without limitation personal computers, servers, smart phones, media players, electronic tablets, game consoles, email devices, etc. In some implementations, server device 800 can include one or more processors 802, volatile memory 804, non-volatile memory 806, and one or more peripherals 808. These components can be interconnected by one or more computer buses 810.

Processor(s) 802 can use any known processor technology, including but not limited to graphics processors and multi-core processors. Suitable processors for the execution of a program of instructions can include, by way of example, both general and special purpose microprocessors, and the sole processor or one of multiple processors or cores, of any kind of computer. Bus 810 can be any known internal or external bus technology, including but not limited to ISA, EISA, PCI, PCI Express, USB, Serial ATA, or FireWire. Volatile memory 804 can include, for example, SDRAM. Processor 802 can receive instructions and data from a read-only memory or a random-access memory or both. Essential elements of a computer can include a processor for executing instructions and one or more memories for storing instructions and data.

Non-volatile memory 806 can include by way of example semiconductor memory devices, such as EPROM, EEPROM, and flash memory devices; magnetic disks such as internal hard disks and removable disks; magneto-optical disks; and CD-ROM and DVD-ROM disks. Non-volatile memory 806 can store various computer instructions including operating system instructions 812, communication instructions 814, application instructions 816, and application data 817. Operating system instructions 812 can include instructions for implementing an operating system (e.g., Mac OS®, Windows®, or Linux). The operating system can be multi-user, multiprocessing, multitasking, multithreading, real-time, and the like. Communication instructions 814 can include network communications instructions, for example, software for implementing communication protocols, such as TCP/IP, HTTP, Ethernet, telephony, etc. Application instructions 816 can include instructions for integral generation and other analysis according to the systems and methods disclosed herein. For example, application instructions 816 can include instructions for components 104-118 described above in conjunction with FIG. 1. Application data 817 can include data corresponding to 104-118 described above in conjunction with FIG. 1.

Peripherals 808 can be included within server device 800 or operatively coupled to communicate with server device 800. Peripherals 808 can include, for example, network subsystem 818, input controller 820, and disk controller 822. Network subsystem 818 can include, for example, an Ethernet of WiFi adapter. Input controller 820 can be any known input device technology, including but not limited to a keyboard (including a virtual keyboard), mouse, track ball, and touch-sensitive pad or display. Disk controller 822 can include one or more mass storage devices for storing data files; such devices include magnetic disks, such as internal hard disks and removable disks; magneto-optical disks; and optical disks.

FIG. 9 is an example computing device that can be used within the system 100 of FIG. 1, according to an embodiment of the present disclosure. In some embodiments, device 900 can be any of client devices 102a-n. The illustrative user device 900 can include a memory interface 902, one or more data processors, image processors, central processing units 904, and/or secure processing units 905, and peripherals subsystem 906. Memory interface 902, one or more central processing units 904 and/or secure processing units 905, and/or peripherals subsystem 906 can be separate components or can be integrated in one or more integrated circuits. The various components in user device 900 can be coupled by one or more communication buses or signal lines.

Sensors, devices, and subsystems can be coupled to peripherals subsystem 906 to facilitate multiple functionalities. For example, motion sensor 910, light sensor 912, and proximity sensor 914 can be coupled to peripherals subsystem 906 to facilitate orientation, lighting, and proximity functions. Other sensors 916 can also be connected to peripherals subsystem 906, such as a global navigation satellite system (GNSS) (e.g., GPS receiver), a temperature sensor, a biometric sensor, magnetometer, or other sensing device, to facilitate related functionalities.

Camera subsystem 920 and optical sensor 922, e.g., a charged coupled device (CCD) or a complementary metal-oxide semiconductor (CMOS) optical sensor, can be utilized to facilitate camera functions, such as recording photographs and video clips. Camera subsystem 920 and optical sensor 922 can be used to collect images of a user to be used during authentication of a user, e.g., by performing facial recognition analysis.

Communication functions can be facilitated through one or more wired and/or wireless communication subsystems 924, which can include radio frequency receivers and transmitters and/or optical (e.g., infrared) receivers and transmitters. For example, the Bluetooth (e.g., Bluetooth low energy (BTLE)) and/or WiFi communications described herein can be handled by wireless communication subsystems 924. The specific design and implementation of communication subsystems 924 can depend on the communication network(s) over which the user device 900 is intended to operate. For example, user device 900 can include communication subsystems 924 designed to operate over a GSM network, a GPRS network, an EDGE network, a WiFi or WiMax network, and a Bluetooth™M network. For example, wireless communication subsystems 924 can include hosting protocols such that device 900 can be configured as a base station for other wireless devices and/or to provide a WiFi service.

Audio subsystem 926 can be coupled to speaker 928 and microphone 930 to facilitate voice-enabled functions, such as speaker recognition, voice replication, digital recording, and telephony functions. Audio subsystem 926 can be configured to facilitate processing voice commands, voice-printing, and voice authentication, for example.

I/O subsystem 940 can include a touch-surface controller 942 and/or other input controller(s) 944. Touch-surface controller 942 can be coupled to a touch-surface 946. Touch-surface 946 and touch-surface controller 942 can, for example, detect contact and movement or break thereof using any of a plurality of touch sensitivity technologies, including but not limited to capacitive, resistive, infrared, and surface acoustic wave technologies, as well as other proximity sensor arrays or other elements for determining one or more points of contact with touch-surface 946.

The other input controller(s) 944 can be coupled to other input/control devices 948, such as one or more buttons, rocker switches, thumb-wheel, infrared port, USB port, and/or a pointer device such as a stylus. The one or more buttons (not shown) can include an up/down button for volume control of speaker 928 and/or microphone 930.

In some implementations, a pressing of the button for a first duration can disengage a lock of touch-surface 946; and a pressing of the button for a second duration that is longer than the first duration can turn power to user device 900 on or off. Pressing the button for a third duration can activate a voice control, or voice command, module that enables the user to speak commands into microphone 930 to cause the device to execute the spoken command. The user can customize a functionality of one or more of the buttons. Touch-surface 946 can, for example, also be used to implement virtual or soft buttons and/or a keyboard.

In some implementations, user device 900 can present recorded audio and/or video files, such as MP3, AAC, and MPEG files. In some implementations, user device 900 can include the functionality of an MP3 player, such as an iPod™. User device 900 can, therefore, include a 36-pin connector and/or 8-pin connector that is compatible with the iPod. Other input/output and control devices can also be used.

Memory interface 902 can be coupled to memory 950. Memory 950 can include high-speed random access memory and/or non-volatile memory, such as one or more magnetic disk storage devices, one or more optical storage devices, and/or flash memory (e.g., NAND, NOR). Memory 950 can store an operating system 952, such as Darwin, RTXC, LINUX, UNIX, OS X, Windows, or an embedded operating system such as VxWorks.

Operating system 952 can include instructions for handling basic system services and for performing hardware dependent tasks. In some implementations, operating system 952 can be a kernel (e.g., UNIX kernel). In some implementations, operating system 952 can include instructions for performing voice authentication.

Memory 950 can also store communication instructions 954 to facilitate communicating with one or more additional devices, one or more computers and/or one or more servers. Memory 950 can include graphical user interface instructions 956 to facilitate graphic user interface processing; sensor processing instructions 958 to facilitate sensor-related processing and functions; phone instructions 960 to facilitate phone-related processes and functions; electronic messaging instructions 962 to facilitate electronic messaging-related process and functions; web browsing instructions 964 to facilitate web browsing-related processes and functions; media processing instructions 966 to facilitate media processing-related functions and processes; GNSS/Navigation instructions 968 to facilitate GNSS and navigation-related processes and instructions; and/or camera instructions 970 to facilitate camera-related processes and functions.

Memory 950 can store application (or “app”) instructions and data 972, such as instructions for the apps described above in the context of FIGS. 1-7. Memory 950 can also store other software instructions 974 for various other software applications in place on device 900.

The described features can be implemented in one or more computer programs that can be executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language (e.g., Objective-C, Java), including compiled or interpreted languages, and it can be deployed in any form, including as a stand-alone program or as a module, component, subroutine, or other unit suitable for use in a computing environment.

Suitable processors for the execution of a program of instructions can include, by way of example, both general and special purpose microprocessors, and the sole processor or one of multiple processors or cores, of any kind of computer. Generally, a processor can receive instructions and data from a read-only memory or a random-access memory or both. The essential elements of a computer may include a processor for executing instructions and one or more memories for storing instructions and data. Generally, a computer may also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory may be supplemented by, or incorporated in, ASICs (application-specific integrated circuits).

To provide for interaction with a user, the features may be implemented on a computer having a display device such as an LED or LCD monitor for displaying information to the user and a keyboard and a pointing device such as a mouse or a trackball by which the user may provide input to the computer.

The features may be implemented in a computer system that includes a back-end component, such as a data server, or that includes a middleware component, such as an application server or an Internet server, or that includes a front-end component, such as a client computer having a graphical user interface or an Internet browser, or any combination thereof. The components of the system may be connected by any form or medium of digital data communication such as a communication network. Examples of communication networks include, e.g., a telephone network, a LAN, a WAN, and the computers and networks forming the Internet.

The computer system may include clients and servers. A client and server may generally be remote from each other and may typically interact through a network. The relationship of client and server may arise by virtue of computer programs running on the respective computers and having a client-server relationship to each other.

One or more features or steps of the disclosed embodiments may be implemented using an API. An API may define one or more parameters that are passed between a calling application and other software code (e.g., an operating system, library routine, function) that provides a service, that provides data, or that performs an operation or a computation.

The API may be implemented as one or more calls in program code that send or receive one or more parameters through a parameter list or other structure based on a call convention defined in an API specification document. A parameter may be a constant, a key, a data structure, an object, an object class, a variable, a data type, a pointer, an array, a list, or another call. API calls and parameters may be implemented in any programming language. The programming language may define the vocabulary and calling convention that a programmer will employ to access functions supporting the API.

In some implementations, an API call may report to an application the capabilities of a device running the application, such as input capability, output capability, processing capability, power capability, communications capability, etc.

While various embodiments have been described above, it should be understood that they have been presented by way of example and not limitation. It will be apparent to persons skilled in the relevant art(s) that various changes in form and detail may be made therein without departing from the spirit and scope. In fact, after reading the above description, it will be apparent to one skilled in the relevant art(s) how to implement alternative embodiments. For example, other steps may be provided, or steps may be eliminated, from the described flows, and other components may be added to, or removed from, the described systems. Accordingly, other implementations are within the scope of the following claims.

In addition, it should be understood that any figures which highlight the functionality and advantages are presented for example purposes only. The disclosed methodology and system are each sufficiently flexible and configurable such that they may be utilized in ways other than that shown.

Although the term “at least one” may often be used in the specification, claims and drawings, the terms “a”, “an”, “the”, “said”, etc. also signify “at least one” or “the at least one” in the specification, claims and drawings.

Finally, it is the applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

1. A system for data collection during therapy on a patient comprising:
a grip device to be held by the patient, the grip device comprising one or more buttons and being configured to:
detect pressing of a pattern of the one or more buttons by the patient; and
transmit an indication of the pattern to a user device;
a mask device to be positioned over eyes of the patient, the mask device comprising one or more functional near infrared spectroscopy (fNIRS) sensors and being configured to:
measure fNIRS data from the patient; and
transmit the fNIRS data to the user device; and
a wearable device to be worn by the patient, the wearable device being configured to:
measure biometric data from the patient; and
transmit the biometric data to the user device;
wherein the user device is configured to provide a playback interface displaying at least one of the fNIRS data or biometric data.

2. The system of claim 1 further comprising a provider device communicably coupled to the user device and configured to provide the playback interface.

3. The system of claim 1, wherein the biometric data comprises at least one of a heart rate, an average body temperature, motion information, oxygen levels, respiratory rates, blood pressure, or skin moisture information.

4. The system of claim 1, wherein the mask device further comprises at least one microphone and is configured to:
measure audio data from the patient; and
transmit the audio data to the user device to be displayed in the playback interface.

5. The system of claim 4, wherein the user device is configured to generate a transcription based on the audio data and display the transcription in the playback interface.

6. The system of claim 4, wherein the user device is configured to perform vocal analysis and sentiment analysis on the audio data and quantify a mood assessment for the patient.

7. The system of claim 4 further comprising a server communicably coupled to the user device, wherein the server is configured to:
receive the audio data;
generate a transcription based on the audio data; and
transmit the transcription to the user device.

8. The system of claim 4 further comprising a server communicably coupled to the user device, wherein the server is configured to:
receive the audio data;
perform vocal analysis on the audio data;
perform sentiment analysis on the audio data;
quantify a mood assessment for the patient; and
transmit the mood assessment to the user device.

9. The system of claim 1, wherein the playback interface comprises a linear editor and is configured to:
receive a selection of at least one data stream; and
play the at least one selected data stream in synchronization on the user device.

10. The system of claim 9 further comprising a server communicably coupled to the user device, wherein the server is configured to:
receive the biometric data and the fNIRS data; and
analyze the biometric data and the fNIRS data to detect, via a machine learning algorithm, at least one timepoint.

11. The system of claim 9, wherein the user device is configured to detect, via a machine learning algorithm, at least one timepoint in the at least one data stream.

12. The system of claim 11, wherein the playback interface is configured to receive an annotation for the at least one timepoint.

13. The system of claim 1, further comprising a server communicably coupled to the user device, wherein the server is configured to:
receive the biometric data and fNIRS data from the user device;
identify a previously recorded data stream associated with the patient; and
execute a neurofeedback procedure on the received data and the identified data stream.

14. The system of claim 13, wherein the server is configured to, in response to the execution of the neurofeedback procedure, transmit one or more feedback signals to the user device.

15. The system of claim 13, wherein the execution of the neurofeedback procedure is performed based on the pattern of the one or more buttons.

16. The system of claim 1, wherein the user device comprises a data hub configured to store baseline data of the patient and the user device is configured to compare the fNIRS data and biometric data to the baseline data.

17. A method for administering a therapy on a patient comprising:
receiving fNIRS data measured by a mask device positioned over eyes of the patient;
receiving biometric data measured by a wearable device worn by the patient;
receiving audio data measured by at least one microphone positioned at a head of the patient; and
providing a user-configurable playback interface displaying at least one of the fNIRS data or biometric data.

18. The method of claim 17 further comprising analyzing the biometric data and the fNIRS data, via a machine learning algorithm, to detect at least one timepoint.

19. The method of claim 18 further comprising receiving, via the playback interface, an annotation for the at least one timepoint.

20. The method of claim 17 further comprising:
performing vocal analysis on the audio data;
performing sentiment analysis on the audio data;
quantifying a mood assessment for the patient; and
displaying the mood assessment via the playback interface.

21. A device for collecting data from a patient during a therapy comprising:
a microphone configured to record patient audio;
a speaker configured to play audio;
one or more functional near infrared spectroscopy (fNIRS) sensors; and
a light emitting diode (LED) configuration configured to illuminate a pattern based on a received neurofeedback signal.

22. The device of claim 21, wherein the device is configured to be worn around a head of the patient.

23. The device of claim 21 comprising a zippable functionality, wherein the device is configured to operate in conjunction with a virtual reality headset.

24. The device of claim 21 comprising a nebulizer extension for drug delivery and configured to collect dose data.

25. The device of claim 21 comprising one or more pupillometric sensors configured to measure pupil dilation information from the patient.

26. The device of claim 21 comprising one or more electrooculography (EOG) sensors configured to detect eye movement of the patient.

27. The device of claim 21 comprising a camera module configured to record a video feed of a face of the patient.

28. A system for data collection during therapy on a user comprising:
a grip device to be held by the user, the grip device comprising one or more buttons and being configured to:
detect pressing of a pattern of the one or more buttons by the user; and
transmit an indication of the pattern to a user device;
a mask device to be positioned over eyes of the user, the mask device comprising one or more functional near infrared spectroscopy (fNIRS) sensors and a camera module and being configured to:
measure fNIRS data from the user;
record a video feed of the user; and
transmit the fNIRS data and the video feed to the user device;
wherein the user device is configured to provide a playback interface displaying at least one of the fNIRS data or biometric data.

29. The system of claim 28 further comprising a provider device communicably coupled to the user device and configured to provide the playback interface.

30. The system of claim 28, wherein the mask device further comprises at least one microphone and is configured to:
measure audio data from the user; and
transmit the audio data to the user device to be displayed in the playback interface.

31. The system of claim 28, wherein the user device is configured to process the video feed to detect pulse data of the user.

32. The system of claim 28, wherein the user device is configured to, in response to detecting pressing of the pattern of the one or more buttons, bookmark a moment in a user data stream.

33. A method for administering a therapy on a user comprising:
receiving fNIRS data measured by a mask device positioned over eyes of the user;
receiving a video feed recorded by the mask device;
processing the video feed to detect pulse data of the user;
receiving audio data measured by at least one microphone positioned at a head of the user; and
providing a user-configurable playback interface displaying at least one of the fNIRS data, the audio data, or the pulse data.

34. The method of claim 33 further comprising receiving, via the playback interface, an annotation for at least one section of a data stream, the data stream comprising at least one of the fNIRS data, the audio data, or the pulse data.

35. The method of claim 33 further comprising receiving an annotation for at least one section of a data stream, the data stream comprising at least one of the fNIRS data, the audio data, or the pulse data from a provider device.

36. The method of claim 33 further comprising receiving an integral from a provider decide on a pre-defined schedule.
